(19) United States
(12) Patent Application Publication
(10) Pub. No.: US 2025/0375658 A1
(43) Pub. Date: Dec. 11, 2025

(54) LUMBAR-PELVIC TRAINING SYSTEM

(71) Applicant: National Taiwan University, Taipei (TW)

(72) Inventors: Wei-Li HSU, Taipei (TW); Shiu-Min WANG, Taipei (TW)

(21) Appl. No.: 18/738,770

(22) Filed: Jun. 10, 2024

(51) Int. Cl. A63B 24/00; A63B 23/02

(52) U.S. Cl. CPC A63B 24/0006; A63B 23/0233; A63B 2024/0012

(57) ABSTRACT

The present invention provides a lumbar-pelvic training system and a lumbar-pelvic training method, wherein the system comprises: a sensing unit configured to detect a user's movement changes in real time to generate at least one movement information; a processing unit configured to acquire and analyze the movement information, and generate at least one control signal; a feedback unit configured to receive the control signal and provide at least one real-time feedback for the user based on said control signal; and a display unit configured to display the real-time feedback. The lumbar-pelvic training system detects the user's lumbar-pelvic movements and provides real-time visual guidance and adjustment suggestions, thereby allowing the user to monitor his/her own training status, and improving training efficiency and accuracy.

### FIELD OF THE INVENTION

The present invention relates to a system and method for training lumbar-pelvic, and more particularly, a lumbar-pelvic training system that provides real-time feedback to encourage proper lumbar-pelvic position. The present invention focuses on improving lumbar-pelvic control skills in sports activities by assessing a user's lumbar-pelvic movements through real-time sensing technology to provide real-time feedback and visual guidance.

### BACKGROUND OF THE INVENTION

Baseball is a team sport in which a pitcher throws a baseball, a batter hits, and a catcher catches to score runs. Baseball involves the transfer of power from the lower limbs to the upper limbs to accomplish the action of pitching or hitting. Different grips and pitching postures create different ball paths, and it's one of the most popular sports among many people due to its versatility.

As more and more people become involved in baseball, training for baseball becomes more common. However, baseball is a whole-body sport, and incorrect posture is likely to cause injuries. Nonetheless, the training of pitching posture is based on the naked eye observation of the coach, and it is impossible to accurately acquire the force exerted by the muscles and joints. In addition, the movement of pitching focuses on the lumbar-pelvic control of the lumbar-iliac regions, and it should be well controlled, or it might lead to incorrect power transfer and more consumption of arm strength, which may not only increases the burden on the arm but may also cause injuries.

Furthermore, In addition to baseball, many other sports involve the lumbar-pelvic movement, such as golf, tennis or the like. Therefore, the importance of lumbar-pelvic control is increasing for athletes in various fields. As such, there is a great need for an precise and real-time lumbar-pelvic training system/method in this field.

### SUMMARY OF THE INVENTION

The summary of the invention aims to provide a simplified summary of the disclosure, so that the reader has a basic understanding of the disclosure. This summary of the invention is not a complete overview of the disclosure, and it is not intended to point out important/critical elements of embodiments of the invention or define the scope of the invention.

In view of the problems to be solved and the background of the art, the present invention provides a unique training system that provides real-time visual feedback of a user's lumbar-pelvic control and precise guidance and adjustments so that the user can quickly master the skills and performance, thereby enhancing the efficiency and accuracy of the training.

The primary objective of the present invention is to provide a lumbar-pelvic training system, comprising: a sensing unit configured to detect a user's movement changes in real time to generate at least one movement information; a processing unit configured to acquire and analyze the movement information, and generate at least one control signal; a feedback unit configured to receive the control signal and provide at least one real-time feedback for the user based on said control signal; and a display unit configured to display the real-time feedback.

In one aspect of the present invention, said sensing unit comprises at least one inertial measurement unit (IMU) sensor.

In one aspect of the present invention, said movement information comprises a lumbar-pelvic angle.

In one aspect of the present invention, the lumbar-pelvic training system further comprising a memory unit in communication with the processing unit, configured to store the user's movement.

In one aspect of the present invention, said display unit comprises a screen.

In one aspect of the present invention, said real-time feedback comprises a visual movement guidance.

Another objective of the present invention is to provide a lumbar-pelvic training method comprising the following steps: (a) configuring a sensing unit on or corresponding to an user; (b) detecting the user's movement changes to generate at least one movement information via the sensing unit; (c) acquiring and analyzing the movement information to generate at least one control signal via a processing unit; (d) receiving the control signal and providing at least one real-time feedback for the user based on said control signal via a feedback unit; (e) displaying the real-time feedback via a display unit.

In one aspect of the present invention, said user's movement includes single-leg bridge exercise, cat and dog exercise, lateral slide exercise, single-leg squat exercise, or combinations thereof.

In one aspect of the present invention, said user's movement is a whole-body movement or a partial-body movement.

In one aspect of the present invention, said movement information is a lumbar-pelvic angle.

In one aspect of the present invention, analyzing the movement information to generate the control signal in step (c) is determined by comparing the movement information to a lumbar-pelvic angle threshold range.

In one aspect of the present invention, said movement information is within the lumbar-pelvic angle threshold range, and then a positive real-time feedback is provided.

In one aspect of the present invention, said movement information falls outside the lumbar-pelvic angle threshold range, and then a negative real-time feedback is provided.

The lumbar-pelvic training system of the present invention is capable of accurately detecting and evaluating the user's lumbar-pelvic control situation by means of advanced sensing technology, so as to enable the user to acquire the accuracy of his/her own movements and to provide real-time visual guidance and adjustment, thereby enhancing the user's training efficiency and helping obtaining a better sports performance. In addition, the system of the present invention has a wide market demand and commercial potential, as it not only meets the training needs of professional athletes, but also serves as a training system for amateur athletes or other sports enthusiasts.

After referring to the following embodiments, those with ordinary knowledge in the technical field to which the present invention pertains to can easily understand the basic spirit of the present invention and its purpose, as well as the technical means and implementation aspects adopted by the present invention.

### BRIEF DESCRIPTION OF THE DRAWINGS

In order to make the above and other objects, features, advantages and embodiments of the present invention more obvious and understandable, the drawings are described as follows:

FIG. 1 illustrates the lumbar-pelvic training system of one of the embodiments of the present invention.

FIG. 2A to 2D illustrate schematic diagram of lumbar-pelvic training movement.

### DETAILED DESCRIPTION OF THE INVENTION

In this section, the contents of the present invention will be described in detail through the following examples. These examples are for illustration only, and those skilled in the art can easily think of various modifications and changes. Various embodiments of the present invention will be described in detail below. In this specification and the appended patent applications, unless the context clearly indicates otherwise, “a” and “the” can also be interpreted as plural.

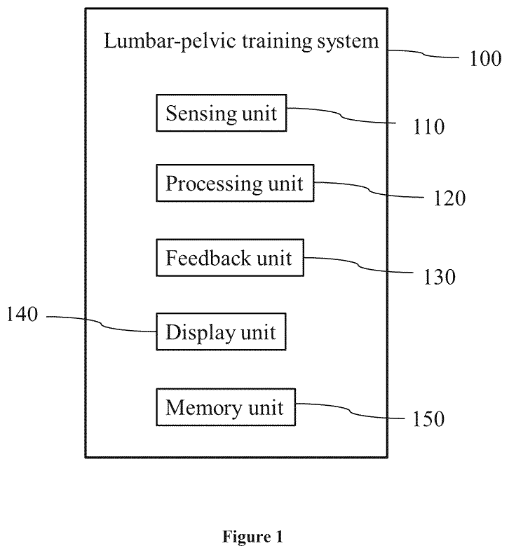

As seen in FIG. 1, the present invention provides a lumbar-pelvic training system 100, comprising: a sensing unit 110 configured to detect a user's movement changes in real time to generate at least one movement information; a processing unit 120 configured to acquire and analyze the movement information, and generate at least one control signal; a feedback unit 130 configured to receive the control signal and provide at least one real-time feedback for the user based on said control signal; and a display unit 140 configured to display the real-time feedback.

In an embodiment of the present invention, the sensory unit 110 comprises at least one inertial measurement unit (IMU) sensor. In detail, the inertial measurement unit comprises plural inertial sensors which are separated; e.g., for disposing at different body parts such as the lumbar spine, the pelvic of the user. More specifically, the inertial sensors include, for example but not limited to, at least one three-axis gyroscope; more preferably, said inertial sensors include at least one three-axis gyroscope and at least one accelerometer. By means of the inertial measurement unit of the sensing unit 110, the present system measures the lumbar-pelvic angle in order to generate an inertia measurement value, which is transmitted as a movement information to an processing unit 120. In an embodiment of the present invention, said movement information comprises, but not limited to, a lumbar-pelvic angle, a body swing, etc.; in a preferred embodiment, the movement information comprises a lumbar-pelvic angle.

In an embodiment of the present invention, the processing unit 120 can be various processors, central processing units, microprocessors or other computing devices known to those with ordinary skill in the technical field to which the present invention belongs.

In an embodiment of the present invention, the feedback unit 130 generates a real-time feedback after receiving the control signal, which real-time feedback includes, but not limited to, visual movement guidance, audible prompt, posture adjustment suggestions, etc.; in a preferred embodiment, the real-time feedback comprises a visual movement guidance.

As used herein, real-time feedback is defined as a quick and appropriate response based on the posture presented by the user, which includes, but not limited to, postural adjustments, lumbar-pelvic angle, body rotation angle or the like.

In a preferred embodiment of the present invention, the feedback unit 130 provides an adaptive prescription according to the user's performance, and gives different restriction ranges according to their different movements to increase the difficulty of training. Specifically, the present system provides different training methods depending on the level of the user, such as limiting the range of lumbar-pelvic angles, or the amplitude of body sway or the like, in order to provide personalized training to meet different exercise needs. In a preferred embodiment of the present invention, the feedback unit 130 further comprises a sound emitter or light emitter. When the user's movements do not conform to the standard posture, the feedback unit 130 prompts the user via sound or light until the user's movements are standardized.

In an embodiment of the present invention, said display unit 140 comprises a screen. The visual real-time feedback is displayed on the screen and the user can further adjust their posture according to the feedback through the screen.

In a preferred embodiment of the present invention, the lumbar-pelvic training system 100 further comprises a memory unit 150 in communication with the processing unit 120, configured to store the user's movement. The memory unit 150 can be used to store information about the user's posture, real-time feedback, and to record changes in the user's training. In addition, the memory unit 150 can also store physiotherapy knowledge, sports injury prevention methods, and different training postures to form a database that provides the user with a complete training concept.

Another objective of the present invention is to provide a lumbar-pelvic training method comprising the following steps: (a) configuring a sensing unit on or corresponding to an user; (b) detecting the user's movement changes to generate at least one movement information via the sensing unit; (c) acquiring and analyzing the movement information to generate at least one control signal via a processing unit; (d) receiving the control signal and providing at least one real-time feedback for the user based on said control signal via a feedback unit; (e) displaying the real-time feedback via a display unit.

In a preferred embodiment of the present invention, after step (a), a further step comprises a calibration step, in which the user adopts a prepared posture, the sensor generates an inertial measurement value based on the aforementioned posture, and the angle of rotation is calibrated to zero based on the inertial measurement value. Preferably, the prepared posture is an upright standing posture.

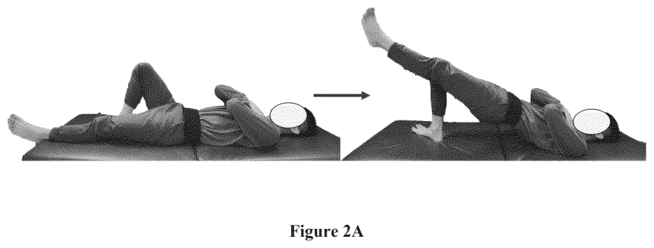

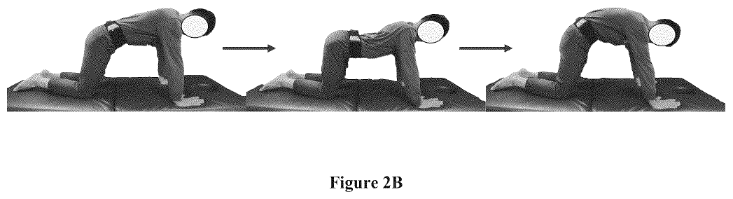

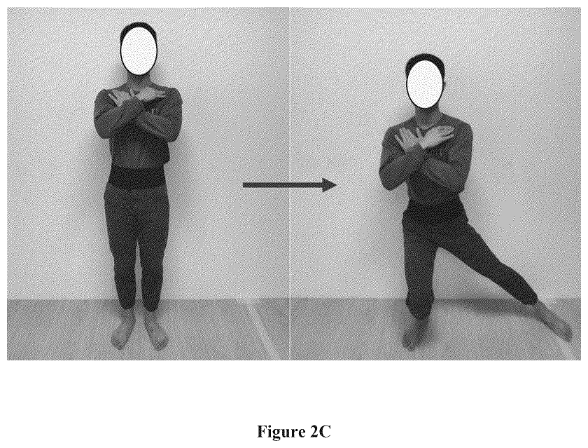

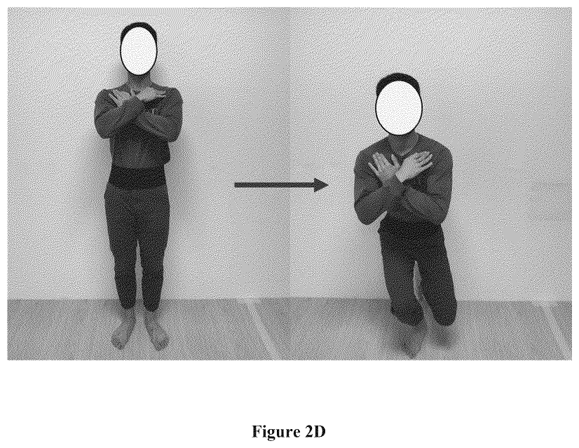

As seen in FIG. 2A to 2D. In an embodiment of the present invention, the user's movement is a whole-body movement or a partial-body movement. More specifically, the user's movement includes single-leg bridge exercise (FIG. 2A), cat and dog exercise (FIG. 2B), lateral slide exercise (FIG. 2C), single-leg squat exercise (FIG. 2D), or combinations thereof.

In an embodiment of the present invention, said movement information comprises, but not limited to, a lumbar-pelvic angle, a body swing, etc.; in a preferred embodiment, said movement information is a lumbar-pelvic angle.

In an embodiment of the present invention, analyzing the movement information to generate the control signal in step (c) is determined by comparing the movement information to a lumbar-pelvic angle threshold range. Furthermore, in an embodiment of the present invention, said movement information is within the lumbar-pelvic angle threshold range, and then a positive real-time feedback is provided; in another embodiment of the present invention, said movement information falls outside the lumbar-pelvic angle threshold range, and then a negative real-time feedback is provided. Said positive real-time feedback refers to the user's movement in a standardized posture, and therefore the feedback unit presents a real-time angle indicating that the user's posture is correct, whereas said negative real-time feedback refers to the user's movement not in a standardized posture, and therefore the feedback unit further presents a warning sign to instruct the user to adjust his/her movement to the correct posture. The warning sign may be, but not limited to, light, sound, vibration, etc. to remind the user to adjust his/her lumbar spine and pelvic to the proper angle.

The real-time angle refers to that when the user stands in front of the display unit, the display unit generates an angle according to the user's posture change, and the angle varies with the user's posture change.

The specific lumbar-pelvic training method of the present invention is as follows: a sensor is set on the user's lumbar-pelvic, calibrated first, and after the rotation angle is zeroed, the user is made to perform single-leg bridge exercise, cat and dog exercise, lateral slide exercise, single-leg squat exercise or the like. The lumbar-pelvic angle is measured by the inertial measurement unit in the sensor, which generates movement information; and the processing unit collects and analyzes the movement information. The analysis is performed by comparing the difference between the movement information and the threshold of the standard lumbar-pelvic angle. When the movement information falls within the lumbar-pelvic angle threshold range, a positive real-time feedback is provided to show the immediate angle and to indicate a correct posture; when the movement information fails to fall within the lumbar-pelvic angle threshold range, a negative real-time feedback is provided to indicate an incorrect posture with a warning sign. The feedback unit receives the real-time feedback and transmits it to the display unit, where the result is shown on the screen of the display unit. As such, the user can adjust his/her training posture according to the feedback to obtain the correct posture.

### SPECIFIC EXAMPLES

The example is designed to enable participants to perform lumbar-pelvic training utilizing the lumbar-pelvic training system of the present invention. Each participant wears an IMU securely attached to an elastic belt worn around the waist, covering the anterior and posterior superior iliac spines. Additionally, the real-time visual biofeedback with pelvic joint angle will be displayed on the screen in front of them.

Calibration is performed before the start of training: the participants stand in an upright position, meanwhile the IMU generates an inertia measurement value based on the aforementioned postures, and the rotation angle is zeroed based on these inertia measurement values.

The training system incorporates four specific training movements: 1) cat and dog exercise; 2) single leg bridge exercise; 3) lateral slide exercise; 4) single leg squat exercise. The lumbar-pelvic angle varied with different movements; wherein the standard lumbar-pelvic threshold range was set according to these movements, and the following movements were continued to measure the change of lumbar-pelvic angle.

### Example 1: Cat and Dog Exercise

In cat and dog exercise, participants begin in the all-four position and then take instructions from trainers to tilt their pelvis forward or backward to specific angles. They will perform 10 repetitions of tilting their pelvis forward and 10 repetitions of tilting it backward according to the instructions. Participants are able to adjust the angles of their movements based on real-time visual feedbacks with pelvic joint angles.

### Example 2: Single Leg Bridge Exercise

In single leg bridge exercise, participants lie flat on a typical treatment table in a supine posture during the test. They will flex their test knee and hip while placing their foot on the table. The hip and knee on the non-test leg will be kept at rest. Participants will be instructed to cross their arms and position them in front of their chests. They will lift their pelvis off the table and perform the bridge technique, flexing the test knee to about 90 degrees while extending the test hip. Simultaneously, the non-test knee will be fully extended to align with the trunk. They will be required to maintain this static position for 30 seconds. Participants can adjust the angles of their pelvic movements based on real-time visual feedbacks with pelvic joint angles, thereby limiting rotational and anterior-posterior tilt within a range of 3 degrees. The test is repeated five times on each side.

### Example 3: Lateral Slide Exercise

In lateral slide exercise, each participant performs a single-leg squat with a lateral slide while maintaining a flexed stance knee at 90 degrees and an upright trunk position. Participants will cross their arms and place them in front of their chests. In the first week, the farthest distance each participant can slide outward will be measured and used as the training distance for the first two weeks. Then, every two weeks, the distance will be increased by 5 cm. The sliding distance of each participant must reach the specified distance. The lateral trunk tilt angle is limited within 30 degrees. Participants can adjust their trunk tilt angle based on real-time visual feedback with pelvic joint angles. Each leg will be trained for 20 repetitions.

### Example 4: Single Leg Squat Exercise

In single leg squat exercise, participants have to complete squats while maintaining their balance and upright torsos. In the meantime, they also need to cross their arms over their chest and flex their non-standing knee to 90 degrees. The forward inclination angle of the trunk is limited initially to less than 30 degrees for the first two weeks, and the upper limit will be reduced by 5 degrees for every two weeks. Participants will be able to adjust their trunk angle based on real-time visual feedback with pelvic joint angles. Each leg will be trained for 20 repetitions.

A movement information is generated upon completion of the above exercises and is collected and analyzed. If the absolute value of the lumbar-pelvic angle is less than 5 degrees, for example, less than 5 degrees, less than 4 degrees, less than 3 degrees, less than 2 degrees, or less than 1 degree, the participants' movement is determined to be correct. If the difference is outside the aforementioned range, the participants will be instructed to adjust to the correct posture. Therefore, the user can obtain proper lumbar-pelvic position more precisely and more efficiently.

The lumbar-pelvic training system/method of the present invention is capable of accurately detecting and evaluating the user's lumbar-pelvic control situation by means of advanced sensing technology, so as to enable the user to acquire the accuracy of his/her own movements and to provide real-time visual guidance and adjustment, thereby enhancing the user's training efficiency and helping obtaining a better sports performance. In addition, the system of the present invention has a wide market demand and commercial potential, as it not only meets the training needs of professional athletes, but also serves as a training device for amateur athletes or other sports enthusiasts.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A lumbar-pelvic training system, comprising:
a sensing unit configured to detect a user's movement changes in real time to generate at least one movement information;
a processing unit configured to acquire and analyze the movement information, and generate at least one control signal;
a feedback unit configured to receive the control signal and provide at least one real-time feedback for the user based on said control signal; and
a display unit configured to display the real-time feedback.

2. The system of claim 1, wherein the sensing unit comprises at least one inertial measurement unit (IMU) sensor.

3. The system of claim 1, wherein the movement information comprises a lumbar-pelvic angle.

4. The system of claim 1, further comprising a memory unit in communication with the processing unit, configured to store the user's movement.

5. The system of claim 1, wherein the display unit comprises a screen.

6. The system of claim 5, wherein the real-time feedback comprises a visual movement guidance.

7. A lumbar-pelvic training method comprising the following steps:
(a) configuring a sensing unit on or corresponding to an user;
(b) detecting the user's movement changes to generate at least one movement information via the sensing unit;
(c) acquiring and analyzing the movement information to generate at least one control signal via a processing unit;
(d) receiving the control signal and providing at least one real-time feedback for the user based on said control signal via a feedback unit;
(e) displaying the real-time feedback via a display unit.

8. The method of claim 7, wherein the user's movement includes single-leg bridge exercise, cat and dog exercise, lateral slide exercise, single-leg squat exercise, or combinations thereof.

9. The method of claim 8, wherein the user's movement is a whole-body movement or a partial-body movement.

10. The method of claim 7, wherein the movement information is a lumbar-pelvic angle.

11. The method of claim 10, wherein analyzing the movement information to generate the control signal in step (c) is determined by comparing the movement information to a lumbar-pelvic angle threshold range.

12. The method of claim 11, wherein the movement information is within the lumbar-pelvic angle threshold range, and then a positive real-time feedback is provided.

13. The method of claim 11, wherein the movement information falls outside the lumbar-pelvic angle threshold range, and then a negative real-time feedback is provided.
